(19) United States
(12) Patent Application Publication
(10) Pub. No.: US 2007/0249725 A1
(43) Pub. Date: Oct. 25, 2007

(54) Pharmaceutical formulations of sympathetic antagonist for the treatment of chronic muscle pain

(76) Inventor: David R. Hubbard, Poway, CA (US)

Correspondence Address: DUANE MORRIS LLP, 101 WEST BROADWAY, SUITE 900, SAN DIEGO, CA, 92101-8285, US

(21) Appl. No.: 11/707,744

(22) Filed: Feb. 16, 2007

Related U.S. Application Data
(60) Provisional application No. 60/774,776, filed on Feb. 17, 2006.

(51) Int. Cl. A61K 31/135; A61P 21/00

(52) U.S. Cl. 514/651

(57) ABSTRACT

The invention encompasses novel pharmaceutical compositions and dosage forms of sympathetic adrenergic antagonists that are formulated for parenteral administration to a human. Also provided are methods of treating a human with the pharmaceutical compositions and dosage forms, and kits of the pharmaceutical compositions and dosage forms.

This application claims priority to U.S. Provisional Application No. 60/774,776 filed Feb. 17, 2007.

### BACKGROUND

It is estimated that up to 20% of the adult population in the United States suffers from chronic and recurrent muscle pain. Each year chronic muscle pain affects 24 million Americans and results in 4.1 million hospitalizations, 3.75 million operations (11.5% of all operations), and 30 million office visits.

Approximately 21 million persons suffer low back pain per year, resulting in a total annual cost of between $20-56 billion. The lifetime prevalence of low back pain lasting 1 or more days of the American population is 56%, 70% of whom consulted physicians. Of those patients with low back pain lasting more that 2 weeks, 31% are hospitalized, and 11.6% undergo surgery. Only 1% of lower back pain sufferers have surgical lumbar disk disease; surgery to remedy this, condition is associated with a 15-30% failure rate, where return to the same occupation is used as a measure of the outcome. The principal reason for failure is incorrect diagnosis. Ironically, psychologic evaluation seems more predictive of a successful outcome following surgery than the current diagnostic studies or surgical findings.

The prevalence of other neuromuscular disorders besides back pain is 53%. Of that percentage, headaches account for 73% and temporomandibular disorders for 12.3%. Both of these conditions are primarily muscular in nature. The total annual cost of headaches alone is $16 billion, while the total annual cost for all other neuromuscular disorders is $11 billion.

Chronic muscle pain, which may comprise pain associated with headaches, neck and lower back problems, fibromyalgia, temporomandibular disorders and myofascial pain syndromes, has been described as epidemic (Sola, A. and Bonica, J. (1990) Management of Pain, Philadelphia, Lea & Febiger). One common type of chronic muscle pain, myofascial pain syndrome, is a neuromuscular dysfunction of skeletal muscle fibers manifested by neuromuscular abnormalities. Examples of such neuromuscular abnormalities include trigger point (TrP) and tender point (TeP) phenomena of various origins. Myofascial pain syndrome is a neuromuscular dysfunction of skeletal muscle fibers manifested by trigger point (TrP) phenomena of various origins and referred phenomena.

The underlying pathophysiology of TrPs has not been conclusively established. However, specific criteria have been defined for identifying TrPs (Simons, D., (1990) Adv. Pain Res. Ther. 17:1:41; Travell et al. Myofascial Pain and Dysfunction, the Trigger Point Manual, New York; Williams & Wilkins (1983); 5-44. These criteria include a palpable firm area of muscle, referred to as the taut band; within the taut band, a localized spot of exquisite tenderness to manual pressure, the TrP; a characteristic pattern of pain, tingling, or numbness in response to sustained pressure on the TrP within the taut band; and a local twitch of the taut band when the TrP is distorted transversely. Although the taut band may be several centimeters long, the TrP itself is reported to be only a few millimeters in diameter. The referral phenomena include referred pain, referred tenderness, or referred autonomic phenomena, such as vasoconstriction, coldness, sweating, pilomotor response, ptosis and hypersecretion.

There are both active TrPs that cause clinical pain syndromes, and latent TrPs, which are painless, and not associated with clinical pain syndromes. Latent TrPs are, like active ones, identified by manual palpation of taut bands, tenderness, and characteristic referral pattern of pain in response to sustained manual pressure. Fifty percent of asymptomatic persons have latent TrPs on examination of the shoulder-girdle musculature. Sola et al. (1955) Am. J. Phys. Med. 34:585-90. TrPs can also be distinguished from Tender points (TePs). Tender points are areas of tenderness that may or may not be in muscle tissue, do not have palpable taut bands, and do not refer pain to adjacent areas. Travell et al., Myofascial Pain and Dysfunction, The Trigger Point Manual, New York; Williams & Wilkins (1983), 5-44.

Patients with a variety of chronic muscle pain syndromes, including tension headaches, neck and lower back problems, fibromyalgia and myofascial pain syndromes present with TrPs in their muscles. An efficacious method of identification and treatment of chronic muscle pain is needed, particularly a treatment which provides long-lasting and/or permanent pharmacologic blockade of trigger point pain and related symptoms. It therefore also is of interest to determine whether there is spontaneous electromyography (EMG) activity in TrPs, and to monitor and eliminate the cause of this EMG activity as a means for devising treatment regimens for chronic muscle pain.

Muscle biopsy studies of TrPs have searched for areas of tissue damage (Yunus, et al. (1981) Semin. Arthritis Rheum. 11:151-71), local hypoxia, (Bengtsson, et al. (1986) J. Am. Rheum. Assoc. 29(7):817:21; Bennett, R. M. (1989) J. Rheum. (Suppl. 19) 16:185-91; Boessevain, M., (1991) Pain 45:227-38; Lund, et al. (1986) Scand. J. Rheum. 15:165-73; Simons, D. G. (1988) Arch. Phys. Med. Rehabil. 69:207-12) of sympathetic hyperactivity, (Bengtsson, A. and Henriksson, K. (1989) J. Rheum. (Suppl. 19) 16:144-9) but have not shown consistent abnormalities by light microscopy, histochemistry, or electron microscopy. Yunus, M. and Kalyan-Raman, U. P. (1989) Rheum. Dis. Clin. North Am. 15:115-34.

Needle electromyography (EMG) of painful muscle syndromes has produced variable results. The first was published by Buchtal and Clemmesen in 1940, (Buchtal, F., Clemmesen, S. (1940) Acta. Med. Scand 150:48-66), who concluded that the spontaneous EMG activity they identified arose in proprioceptive receptors. Since then a number of EMG studies have been performed on patients with lumbar disc disease (Elliott, F. A. 1944) Lancet 1:47-9), tension headache (Pozniak-Patewicz, E. (1976) Headache 4:261-6), fibrositis (Arroyo, P. (1966) J. Florida Med. Assoc. 53:29-31; Kraft, et al. (1968) Arc. Phys. Med. Rehabil. 49, I:155-62) fibromyalgia, (Durette, et al. (1991) Am. J. Phys. Med. Rehabil. 70,3:154-6; McBroom, et al. (1988) Clin. J. Pain 4:117-9; Zidar, et al. (1990) Pain 40:249-54) and myofascial taut bands (Dexter, J. R. and Simons, D. S. (1981) Arch. Phys. Med. Rehabil. 62:521-2; Friction, et al. (1985) Arch. Phys. Med. Rehabil. 66:314-16). High-frequency firing from TrPs has been described (Travell (1957) Proc. Rudolf Virchow Med. Soc. 16:128-36), but in the 1983 TrP Manual, Travell and Simons concluded that TrPs showed no resting activity and that any activity seen was either insertional or motor unit activity (Travell and Simons, supra). Intravenous regional sympathetic blockade with guanethidine also has been utilized. (Bengtsson and Bengtsson (1988) Pain 33: 161-167).

Several currently available treatment modalities or methods of treatment for chronic pain associated with myofascial trigger points have been described. One treatment of myofascial trigger points is local injection of the painful region, without localization of a neuromuscular abnormality, with local anesthetics including lidocaine, procaine and bupivacaine. Travell and Simons, supra; Sola, A. E. Trigger Point Therapy in Clinical Procedures in Emergency Medicine (Edited by J. R. Roberts and J. R. Hedges. Philadelphia, W. B. Saunders, 1985, pp 674-686). However, this treatment reportedly has only temporary effects. Also described is the transdermal delivery to a painful region of alpha-adrenergic blocking agents. Campbell, U.S. Pat. No. 5,070,084 and WO 92/14453. Dry-needling (inserting a needle into the trigger point without injecting anesthetic) is advocated (Gunn, C C, (1980) Spine 5(3):279) although injection of local anesthetic reportedly is more effective and less painful. (Cooper, A. L. (1961) Arch. Phys. Med. 43:704; Brav, E. A. and Sigmond, H. (1941) Ann. Intern. Med., 15:840; Frost, F. A., et al. (1980) Lancet 1(3):499-501).

A preferred agent for treatment of chronic muscle pain in the instant invention is phenoxybenzamine hydrochloride. Phenoxybenzamine hydrochloride is also known by the trademark name “Dibenzyline” (SmithKline Beecham Pharmaceuticals). Oral dibenzyline has a labeled indication for use in control of episodes of hypertension and sweating in patients with pheochromocytoma. For these indications, each Dibenzyline capsule contains 10 mg phenoxybenzamine hydrochloride, and the inactive ingredients consist of benzyl alcohol, cetylpyridinium chloride, D&C Red No. 33, FD&C Red No. 3, FD&C Yellow No. 6, gelatin, lactose, sodium lauryl sulfate and trace amounts of other inactive ingredients. See The Physician's Desk Reference (PDR) Electronic Library, International Thomson Publishing, Montvale N.J., (2003, Vol. 2003.1, version 6.00a).

U.S. Pat. Nos. 5,513,661 and 6,009,875 entitled “Use of Sympathetic Blockade for Treatment of Chronic Muscle Pain”, the contents of which are hereby incorporated by reference, describe and claim, for example, methods of reducing chronic muscle pain by injection of an alpha adrenergic antagonist, such as phenoxybenzamine. However, it was noted that irritation of the muscle at the site of the injection can occur (see col. 17, lines 28-30 of U.S. Pat. No. 6,009,875). In addition, it was reported that Dibenzyline had a relatively short shelf life as these preparations usually had to be administered to the subject within approximately 4 hours.

### SUMMARY

Provided herein are improved pharmaceutical formulations of adrenergic antagonists for the treatment of chronic muscle pain. The formulations provided herein are particularly suitable for minimizing adverse side-effects and/or local irritations associated with parenteral administrations, including, for example, localized intramuscular injections, of phenoxybenzamine pharmaceutical compositions in the treatment of muscle pain. In addition, the formulations provided herein have increased stability which provides phenoxybenzamine preparations as a stable liquid in desired dosages such that it has a favorable shelf-life. Furthermore, the formulations provided herein are particularly suitable for achieving a favorable diffusion profile when injected, for example, by intramuscular injection, into a subject in the treatment of chronic muscle pain.

In one aspect, sympathetic adrenergic antagonists are provided as pharmaceutical compositions suitable for parenteral administration. In certain embodiments, pharmaceutical composition comprises, consist essentially of, or consist of phenoxybenzamine or a salt thereof (e.g. phenoxybenzamine hydrochloride) and a non-aqueous excipient that is formulated as a liquid for parenteral administration to a human. In certain preferred embodiments, the pharmaceutical composition is for intramuscular injection, including for local injection into an area of muscle associated with pain in a human. In certain other embodiments, the pharmaceutical composition is for intravascular injection.

The non-aqueous excipient of the pharmaceutical composition may comprise one or more polyethylene glycol, and in certain embodiments the pharmaceutical composition comprises, consist essentially of, or consist of phenoxybenzamine and a polyethylene glycol. In certain further embodiments, the polyethylene glycols have a molecular weight of between about 300 daltons and about 500 daltons, such as polyethylene glycol 400.

In certain embodiments, the pharmaceutical composition provided herein comprises at least 90% by volume of a polyethylene glycol. In certain other embodiments, the pharmaceutical composition provided herein comprises at least 95% by volume of a polyethylene glycol. In another embodiment, the pharmaceutical composition comprises at least 97% by volume of a polyethylene glycol. In yet another embodiment, the pharmaceutical composition comprises at least 99% by volume of a polyethylene glycol. In yet another embodiment, the pharmaceutical composition consist essentially of phenoxybenzamine or a salt thereof and a polyethylene glycol.

In one aspect, the pharmaceutical composition provided herein comprises a mixture of two or more polyethylene glycols wherein each polyethylene glycol has a different molecular weight.

In certain embodiments, said polyethylene glycol has a molecular weight of between about 300 daltons and about 500 daltons. In certain other embodiments, said polyethylene glycol comprises polyethylene glycol 400. In yet another embodiment, the pharmaceutical composition comprises a mixture of polyethylene glycol 400 and another polyethylene glycol having a different molecular weight.

In one aspect, the pharmaceutical composition comprises phenoxybenzamine. In one embodiment, said phenoxybenzamine is phenoxybenzamine hydrochloride.

In another aspect, the pharmaceutical compositions are provided as dosage forms, including single dosage forms and multiple dosage forms. Suitable dosage forms may comprise phenoxybenzamine as a single dosage in an amount of from about 0.5 mg to about 50 mg, from about 0.3 mg to about 10 mg, from about 2 mg to about 10 mg, from about 3 mg to about 7 mg, about 5 mg, about 3 mg, between about 3 mg and about 7 mg, between about 0.1 mg to about 2 mg, between about 0.3 and about 0.8 mg, and about 0.5 mg of phenoxybenzamine. Single dosages may be administered more than once, including to the same location or to different locations. These dosages may be administered, for example, in a dosage volume of between about 0.3 ml to about 3 ml, about 0.3 ml and about 0.7 ml, a dosage volume of about 0.5 ml having a phenoxybenzamine concentration of about 10 mg/ml, and a dosage volume of about 0.3 ml having a phenoxybenzamine concentration of about 10 mg/ml.

In certain preferred embodiments, the above dosages are for intramuscular injection.

The pH of the pharmaceutical compositions may optionally be adjusted to a desired level. In certain aspects, the pharmaceutical composition provided herein further comprises water and/or an acid or base for the adjustment of the pH. In certain embodiments, the pharmaceutical composition comprises less than about 2% water. In other embodiments, the pharmaceutical composition comprises less than about 1% water. In certain other embodiments, the pharmaceutical composition comprise about 0.5% water. In certain further embodiments, the pharmaceutical composition provided herein has a pH of from about 2 to about 4. in other embodiments, the pharmaceutical composition provided herein has a pH of from about 3 to about 4. In yet another embodiment, the pharmaceutical composition has a pH of from about 2.8 to about 3.8. In a further embodiment, the pharmaceutical composition has a pH of about 3 to about 3.5.

In certain embodiments, the pharmaceutical compositions may further comprise an osmotic modulating agent, a preservative, a buffer, stabilizer, emulsifier, or the like.

In one aspect, the pharmaceutical composition provided herein is formulated for optimal tissue distribution.

In other aspect, the pharmaceutical composition provided herein comprises phenoxybenzamine or a salt thereof and an oil that is a liquid at room temperature. In one embodiment, the pharmaceutical composition consist essentially of a phenoxybenzamine or a salt thereof and a pharmaceutically acceptable oil. In another embodiment, said pharmaceutically acceptable oil is selected from the group consisting of peanut oil, sesame oil, and cottonseed oil.

In another aspect, methods of treating a human, including reducing chronic muscle pain are provided. In one embodiment, the method comprises locating at least one myofascial trigger point in a muscle; and contacting the myofascial trigger point with a pharmaceutical composition provided herein such that said chronic pain is reduced.

In another aspect, kits comprising the pharmaceutical compositions in one or more pharmaceutically acceptable container are provided. In certain embodiments, the kits comprise a pharmaceutical composition provided herein, means for containing the pharmaceutical composition, and means for injection the pharmaceutical composition into a human.

### DETAILED DESCRIPTION

The present invention provides novel pharmaceutical compositions and dosage forms of one or more agents which antagonize or block sympathetic transmission and/or function of the affected muscle fibers (e.g. sympathetic adrenegic antagonists) that are formulated for parenteral administration to a human. A non limiting but preferred sympathetic blocking agent is phenoxybenzamine and salts thereof (e.g. phenoxybenzamine hydrochloride).

In one aspect, sympathetic adrenergic antagonists are provided as pharmaceutical compositions suitable for parenteral administration. In certain embodiments, the pharmaceutical compositions are suitable for intramuscular injection or intra-muscle fiber administration. It is desirable for the sympathetic antagonists to be formulated as pharmaceutical composition designed to minimize or eliminate localized pain, irritation, and inflammation. In another aspect, the pharmaceutical compositions may be formulated to optimize delivery to affected muscle fibers. In another aspect, the pharmaceutical compositions are formulated to have a desired rate of diffusion and/or tissue distribution profile at a selected injection site. The composition may have a formulation that has a reduced diffusion within a selected tissue (e.g. muscle) relative to previous phenoxybenzamine formulations known in the art. In certain other embodiments, the pharmaceutical compositions are formulated for administration by subcutaneous injection. In certain other embodiments, the pharmaceutical compositions are formulated for administration by intravenous injection.

In preferred embodiments, liquid pharmaceutical compositions are provided. In certain preferred embodiments, liquid compositions substantially free of water are provided. However, it is contemplated that some embodiments of liquid pharmaceutical compositions are formulated using at least some amount of simple aqueous or saline excipients. For example, in certain preferred embodiments a liquid pharmaceutical compositions may comprise in total less than about 1% water (w/v), less than about 0.7% water (w/v), less than about 0.5% water (w/v), less than about 0.3% water (w/v), less than about 0.1% water (w/v), less than about 0.05% water (w/v), less than about 0.03% water (w/v), less than about 0.01% water (w/v), or less than about 0.001% water (w/v). However, it is contemplated that certain embodiments may comprise more water. For example, pharmaceutical compositions may comprise in total, for example, less than about 90% water (w/v), less than about 80% water (w/v), less than about 70% water (w/v), less than about 60% water (w/v), less than about 50% water (w/v), less than about 40% water (w/v), less than about 30% water (w/v), less than about 25% water (w/v), less than about 20% water (w/v), less than about 15% water (w/v), less than about 10% water (w/v), less than about 9% water (w/v), less than about 8% water (w/v), less than about 7% water (w/v), less than about 6% water (w/v), less than about 5% water (w/v), less than about 4% water (w/v), less than about 3% water (w/v), less and than about 2% water (w/v), and in ranges encompassed by all of the amounts described above.

Illustrative examples of suitable excipients include without limitation alcohols, polyethylene glycols, propylene glycols, glycerin, peanut oil, sesame oil, cottonseed oil, and the like ((including other ethylene glycols (e.g. ethylene glycol, diethylene glycol, triethylene glycol), propylene glycols (e.g. dipropylene glycol, tripropylene glycol, polypropylene glycols (“PPGs”, PPG 425, PPG 725, PPG 1000, PPG 2000, PPG 3000 and PPG 4000), glycerine or glycerol)). Pharmaceutical compositions provided herein may comprise, without limitation, a diluent or excipient in an amount that is greater than or equal to 99% (w/v), greater than or equal to 98% (w/v), greater than or equal to 97% (w/v), greater than or equal to 96% (w/v), greater than or equal to 95% (w/v), greater than or equal to 90% (w/v), greater than or equal to 85% (w/v), greater than or equal to 80% (w/v), greater than or equal to 75% (w/v), greater than or equal to 70% (w/v), greater than or equal to 65% (w/v), greater than or equal to 60% (w/v), greater than or equal to 55% (w/v), greater than or equal to 50% (w/v), greater than or equal to 45% (w/v), greater than or equal to 40% (w/v), greater than or equal to 35% (w/v), greater than or equal to 30% (w/v), greater than or equal to 25% (w/v), greater than or equal to 20% (w/v), greater than or equal to 15% (w/v), greater than or equal to 10% (w/v), greater than or equal to 5% (w/v), and in ranges encompassed by all of the amounts described above.

For example, in certain embodiments, phenoxybenzamine compositions are provided that consist essentially of a polyethylene glycol and phenoxybenzamine or a salt or acid thereof. In certain embodiments, it is preferred that the phenoxybenzamine compositions have substantially no water of alcohol and are formulated as a liquid at room.

In certain preferred embodiments of the pharmaceutical composition, the excipient is a polyethylene glycol. Thus, in certain embodiments, the pharmaceutical composition comprises, consists of, or consists essentially of phenoxybenzamine and a polyethylene glycol. It is preferred that that such phenoxybenzamine and a polyethylene glycol compositions are a liquid at room temperature. One preferred polyethylene glycol is polyethylene glycol 400 (PEG-400), and in further certain embodiments the pharmaceutical composition comprises, consists of, or consists essentially of phenoxybenzamine and PEG-400. PEG-400 is understood to include a non discrete composite of polyethylene molecules that can have sizes or weights other than 400 daltons, for example PEG-400 includes molecules from about 380 to about 420 daltons, and possibly some molecules outside this size range. Illustrative examples of other polyethylene glycols used in certain other embodiments include, for example, polyethylene glycol 200, polyethylene glycol 300, and polyethylene glycol 600. Other polyethylene glycol molecules are possible, including for example those having sizes of between about 350 and 450 daltons, between about 300 and 500 daltons, and between about 200 and 600 daltons. Still higher molecular weight forms of polyethylene glycols are contemplated, including polyethylene glycol 1000, polyethylene glycol 1450, polyethylene glycol 1500, polyethylene glycol 1540, polyethylene glycol 3350, polyethylene glycol 4000, polyethylene glycol 6000, polyethylene glycol 8000, polyethylene glycol 9000, and polyethylene glycol 10,000. Generally, polyethylene glycols having a molecular weight of greater than about 600 daltons are more viscous and are used in combination with other excipients, including lower molecular weight polyethylene glycols and other excipient described herein (e.g. an alcohol), so that the pharmaceutical formulation is a liquid at room temperature.

A polyethylene glycol ester, for example an ester of PEG-400, may be used in certain embodiments of pharmaceutical compositions. “Handbook of Pharmaceutical Excipients” 4th Ed. 2003, Edited by Raymond C. Rowe, Paul J. Sheskey, and Paul J. Weller, Pharmaceutical Press. Illustrative examples of polyethylene glycol esters include, for example, PEG 200 monolaurate; PEG 200 dilaurate; PEG 200 monostearate; PEG 200 distearate; PEG 300 monostearate; PEG 300 distearate; PEG 300 monooleate; PEG 300 dioleate; PEG 400 monolaurate; PEG 400 dilaurate; PEG 400 monostearate; PEG 400 distearate; PEG 400 monooleate; PEG 400 dioleate; PEG 400 ricinoleate; PEG 600 monolaurate; PEG 600 monostearate; PEG 600 monooleate; PEG 600 dioleate; PEG 1000 stearate; PEG 1500 palmitostearate; PEG 4000 stearate; PEG 6000 palmitostearate. Other chemically modified polyethylene glycol molecules are envisioned as excipients in other embodiments.

In another aspect, dosage forms of pharmaceutical compositions comprising alpha adrenergic antagonist are provided. In certain embodiments, pharmaceutical compositions of phenoxybenzamine as a single dosage form are provided where the amount of phenoxybenzamine is from about 1 mg to about 10 mg, from about 2 mg to about 10 mg, from about 3 mg to about 7 mg, from about 3 mg to about 5 mg, and includes single dosages of about 1.0.mg, 2.0 mg, 2.4 mg, 3 mg, 4 mg, 5 mg, 6 mg, 7 mg, 8 mg, 9 mg, and 10 mg. Certain other single dosage pharmaceutical compositions of phenoxybenzamine are in amounts of about 0.01 mg, 0.1 mg, 0.3 mg, 0.5 mg, 10 mg, 15 mg, 20 mg, 25 mg, 50 mg, and 100 mg. A dosage of 10 mg or more of phenoxybenzamine (e.g. 10 mg to 100 mg) can be used, for example when the pharmaceutical compositions are administered for subcutaneous administration. These single dosage forms may also be administered at multiple injection sites in a patient, as well as at the same injection site after a desired time period has lapsed.

In certain embodiments, liquid dosage forms of alpha adrenergic antagonist suitable for parenteral administration, and in particular suitable for intra muscular injections, are provided. Suitable volumes for intramuscular injection include, for example, between about 0.1 ml and about 1.0 ml, between about 0.4 ml and about 0.6 ml, between about 0.3 ml and about 0.7 ml, including about 0.1 ml, 0.2 ml, 0.3 ml, 0.4 ml, 0.5 ml, 0.6 ml, 0.7 ml, 0.8 ml, 0.9 ml and 1.0 ml. While less preferred, lower volumes, including less than 25 μl, 25 μl to 50 μl, 50 μl to 100 μl, may be used for intramuscular administration, and higher volumes, for example exceeding 1 ml, including between about 1 ml to 5 ml, or more than 5 ml, may also be used for various routes of parenteral administration.

The pH of the pharmaceutical compositions may be adjusted to a desired level, for example, to facilitate a decrease in tissue irritation. A suitable pH includes but is not limited to a physiological pH, for example to a pH of between about 7 and 8. In certain preferred embodiments the pH of the composition is greater than about 1.5, greater than 1.7, greater than about 2.0, greater than 2.3, greater than about 2.5, greater than about 3.0, or greater than about 3.2. In certain non-limiting preferred embodiments the pH is between about 3 and about 3.5. In certain non-limiting preferred embodiments the pH is about 3.2 or about 3.3. In certain embodiments, the pH is between about 2 and about 8. In certain embodiments, the pH is between about 2.5 and about 4.5. In certain non-limiting preferred embodiments, the pH is between about 2.8 and about 3.8. The compositions may additionally include a buffer, for example to stabilize the pH to a desired range. The pharmaceutical compositions may comprise salts of phenoxybenzamine, such as phenoxybenzamine hydrochloride. Other salts of phenoxybenzamine, including those known in the art, may be used, including for example other chloride salts and other salts known in the art. See “Handbook of Pharmaceutical Salts: Properties, Selection, and Use”, P. Heinrich Stahl and Camille G. Wermuth (Eds.) (John Wiley & Sons, 2002), the contents of which are hereby incorporated by reference in their entirety. Further contemplated are pharmaceutical compositions that comprise analogs or intermediates of phenoxybenzamine which have adrenergic receptor blocking activity or otherwise have a desired biologically activity.

The sympathetic antagonists may be used either alone or in combination with other antagonists in the pharmaceutical compositions and methods provided herein. These agents may decrease local tenderness or enhance the effect of the sympathetic antagonist. Examples of these agents are local anesthetics (e.g. lidocaine) and cortical steroids (e.g. prednisone).

In another aspect, a method for treating chronic muscle pain are provided. Methods of identifying and treating neuromuscular abnormalities described in U.S. Pat. No. 5,513,661 and U.S. Pat. No. 6,009,875, both incorporated herein by reference in their entirety, may be used in conjunction with the pharmaceutical compositions and dosage forms provided herein. In certain embodiments, the method involves locally delivering to a neuromuscular abnormality with an amount of a pharmaceutical composition described herein sufficient to inhibit activity associated with that neuromuscular activity. The abnormality may occur in muscles of the head, neck, extremity or back, and may be a sympathetically activated muscle fibers. The method for treating chronic muscle pain may also involve locating one or more myofascial trigger points by manual palpation, inserting of an EMG needle, detecting spontaneous EMG activity associated with the trigger point and directly contacting the trigger point with enough adrenergic antagonist to the trigger point to inhibit the spontaneous EMG activity. The localized delivery of these agents reduces or eliminates undesirable side effects commonly associated with the systemic administration of such compounds, including for example dizziness, postural hypotension, or fatigue. Pain relief usually is reported after the first treatment. However, patients having multiple neuromuscular dysfunctions may receive multiple injections either at the same treatment session or in several treatment sessions. Where the delivery is localized rather than systemic, undesirable side effects such as postural hypotension, dizziness, fatigue and other common cardiovascular events are minimized or eliminated. Localized delivery also permits delivery of dosages that might cause severe harm if delivered systemically. Where the delivery is localized rather than systemic, undesirable side effects such as postural hypotension, dizziness, fatigue and other common cardiovascular events are minimized or eliminated. Localized delivery also permits delivery of dosages that might cause severe harm if delivered systemically.

The pharmaceutical compositions can be provided as kits for use in screening or treatment of patients and/or for one or more treatments. A kit may comprise one or more pharmaceutically acceptable containers comprising a pharmaceutical composition provided herein. For example, a kit may comprise i) a pharmaceutical composition described herein, ii) means for containing the pharmaceutical composition, and iii) means for injection the pharmaceutical composition into a human. In some embodiments the kits may comprise: (1) disposable sterile EMG needles, including monopolar or multi-port needles; (2) a hypodermic syringe; and (3) a container of an appropriate agent, which may be further diluted prior to use or provided at the concentration of use. The containers may include one or more dosages. For screening, a reversible sympathetic antagonist agent, e.g., phentolamine, can be used. For treatment, a pharmaceutical composition or dosage form of a irreversible agent, e.g., phenoxybenzamine, is preferred. In another aspect, dosage forms described herein are provided in syringes, contained in sterilized containers, so that the physician may employ the syringes directly, where the syringes will have the desired amount and concentration of agents. A syringe may have a single dosage described herein, or multiple dosages.

When desired, the container having the sympathetic antagonist agent may also contain agents which may be administered in combination with the sympathetic antagonist agent, such in combination with local anesthetics (e.g. lidocaine) and cortical steroids (e.g. prednisone). Where a combination of agents is employed, these agents are present in the containers in the desired proportional amounts. Thus, the kit may have a plurality of syringes containing a sympathetic antagonistic agent of choice in amounts appropriate for screening and/or for therapy. Where the syringes contain the composition for direct use, usually there will be no need for other reagents for use with the method.

The subject methods and compositions find use in the diagnosis and treatment of chronic muscle pain associated with neuromuscular abnormalities such as myofascial trigger points, tender points, sympathetically activated muscle fibers and the like. Chronic muscle pain syndromes which may be amenable to the method of treatment of the subject invention may include fibromyalgia, myofascial pain syndrome, tension headache, temporomandibular joint dysfunction (TMD), neck and low back pain syndromes and the like. The methods and compositions may also be used as a means of screening for patients who may benefit from treatment, particularly by evaluating the efficacy of reversible antagonists such as phentolamine in blocking pain, dysfunction and/or spontaneous EMG activity in neuromuscular abnormalities.

The following Examples are offered by way of illustration and not by way of limitation.

### EXAMPLES

### Example 1

### Efficacy and Dosage Study for Intramuscular Injection of Phenoxybenzamine Hydrochloride

Methods

A total of 326 male and female subjects, approximately 15 to 85 years old with pain of 4-72 month duration were treated with various doses of phenoxybenzamine. If a potential subject had pain that was clearly muscular and clearly not due to arthritis, nerve root compression or other extramuscular cause, the subject was considered a candidate for phenoxybenzamine hydrochloride injection. Trigger points were identified by palpation. A monopolar EMG needle was then inserted into the trigger point. Another monopolar EMG needle was inserted into a nearby nontender fibers of the same muscle. If the trigger point electrode showed electrical activity with an amplitude greater than 4 times the reference electrode, phenoxybenzamine hydrochloride was injected into the trigger point. The treatment was repeated in up to 10-12 trigger points per treatment session. Subjects could return for up to 6 treatment sessions, depending on the extent of their pain. The initial treatment was with a dose of 25 mg of phenoxybenzamine hydrochloride in an injection volume of 2 ml. The dose and volume were gradually decreased; the bulk of subjects were treated with a dose of 3 mg in 0.3 ml. For 318 subjects, data were available for analysis for the doses of phenoxybenzamine hydrochloride used, efficacy and incidence of serious adverse events. The numbers of subjects treated at each dose are summarized in the table below.

TABLE 1
Subjects Treated with Phenoxybenzamine Hydrochloride

| | Phenoxybenzamine dose | | | |
|---|---|---|---|---|
| | 25 mg | 10-12.5 mg | 10 mg | 3 mg |
| Volume of injection | 2 ml | 0.5 ml | 0.5 ml | 0.3 ml |
| Number of sites | 1 | 1 | 1-6 | 1-50 |
| injected | | | | |
| Number of subjects | 16 | 8 | 84 | 218 |
| treated | | | | |

Results

Subjects were asked to quantify, in terms of percent improvement from baseline, how they felt after treatment. These investigations did not include control groups nor were the methods used to define efficacy prospectively defined or standardized. These results are shown in the following table.

TABLE 2
Efficacy of Phenoxybenzamine Hydrochloride

| | Phenoxybenzamine dose | | | |
|---|---|---|---|---|
| | 25 mg | 10-12.5 mg | 10 mg | 3 mg |
| Number of | 16 | 8 | 84 | 178 (Interim |
| subjects | | | | data) |
| Timepoint | % of Subjects with Greater than | | | Mean % |
| | 25% Improvement | | | improvement |
| One week | 40 | — | — | — |
| One month | 57 | 50 | 52 | — |
| Two month | 66 | 83 | 68 | 54.9 |
| Three month | — | — | 66 | — |
| Four month | 53 | 100 | 63 | — |

These results are noteworthy because many of these subjects had been suffering for over 2 years prior to treatment and had received all conventional therapy prior to treatment with phenoxybenzamine hydrochloride. After two months of treatment, the subjects treated with 3 mg of phenoxybenzamine reported a 54.9% improvement. About 15% of subjects reported no improvement in pain.

The frequencies of other adverse events occurring in the first 199 subjects treated, including the first 87 subjects treated with the 3 mg dose, are given in the table below. Results with an additional 131 subjects treated with the 3 mg dose have been similar.

TABLE 3
Frequency of Adverse Events in 199 Treated Subjects

| | Phenoxybenzamine | | | | | | | |
|---|---|---|---|---|---|---|---|---|
| | 10-25 mg | | 10-12.5 mg | | 3 mg | | | |
| | Injections | | | | | | | |
| | single | | multiple | | multiple | | Total | |
| | N | | | | | | | |
| | 23 | % | 89 | % | 87 | % | 199 | |
| Injection site swelling | 6 | 26% | 40 | 45% | 20 | 23% | 66 | 33% |
| Injection site soreness | 8 | (35%) | 33 | (37%) | 18 | (21%) | 59 | (30%) |
| Dizziness | | (0%) | 18 | (20%) | 3 | (3%) | 21 | (11%) |
| Nausea | | (0%) | 16 | (18%) | 2 | (2%) | 18 | (9%) |
| Lightheadedness | 1 | (4%) | 12 | (13%) | | (0%) | 13 | (7%) |
| Headache | | (0%) | 9 | (10%) | 3 | (3%) | 12 | (6%) |
| Bruising at injection site | | (0%) | 10 | (11%) | 1 | (1%) | 11 | (6%) |
| Pain | 6 | (26%) | | (0%) | 4 | (5%) | 10 | (5%) |
| Fatigue | 1 | (4%) | 4 | (4%) | 3 | (3%) | 8 | (4%) |
| Vomiting | | (0%) | 7 | (8%) | | (0%) | 7 | (4%) |
| Hypesthesia | 2 | (9%) | 4 | (4%) | | (0%) | 6 | (3%) |
| Stiffness | 3 | (13%) | 2 | (2%) | 1 | (1%) | 6 | (3%) |
| Syncope | | (0%) | 6 | (7%) | | (0%) | 6 | (3%) |
| Burning | 1 | (4%) | 3 | (3%) | | (0%) | 4 | (2%) |
| Dyspnea | | (0%) | 3 | (3%) | 1 | (1%) | 4 | (2%) |
| Nasal disorder | 2 | (9%) | 2 | (2%) | | (0%) | 4 | (2%) |
| Taste disorder | 1 | (4%) | 2 | (2%) | 1 | (1%) | 4 | (2%) |
| Paresthesia | | (0%) | 3 | (3%) | | (0%) | 3 | (2%) |
| Rash | 1 | 4% | 2 | 2% | | 0% | 3 | 2% |
| Cold sensation | 1 | (4%) | 1 | (1%) | | (0%) | 2 | (1%) |
| Diarrhea | | (0%) | 2 | (2%) | | (0%) | 2 | (1%) |
| Flu syndrome | | (0%) | 1 | (1%) | 1 | (1%) | 2 | (1%) |
| Injection site drainage | | (0%) | 2 | (2%) | | (0%) | 2 | (1%) |
| Swelling | | (0%) | 2 | (2%) | | (0%) | 2 | (1%) |
| Tremor | | (0%) | 1 | (1%) | 1 | (1%) | 2 | (1%) |
| Diaphoresis | | (0%) | 1 | (1%) | | (0%) | 1 | (0%) |
| Dry mouth | | (0%) | 1 | (1%) | | (0%) | 1 | (0%) |
| Dysphagia | 1 | (4%) | | (0%) | | (0%) | 1 | (0%) |
| Epistaxis | 1 | (4%) | | (0%) | | (0%) | 1 | (0%) |
| Fever | | (0%) | 1 | (1%) | | (0%) | 1 | (0%) |
| Hair disorder | | (0%) | | (0%) | 1 | (1%) | 1 | (0%) |
| Lethargy | | (0%) | | (0%) | 1 | (1%) | 1 | (0%) |
| Palpitations | | (0%) | 1 | (1%) | | (0%) | 1 | (0%) |
| Pharyngitis | | (0%) | 1 | (1%) | | (0%) | 1 | (0%) |
| Pruritis | | (0%) | 1 | (1%) | | (0%) | 1 | (0%) |
| Tachycardia | 1 | (4%) | | (0%) | | (0%) | 1 | (0%) |

### Example II

### Efficacy Dosage Study for Intramuscular Injection of Phenoxybenzamine Hydrochloride

Methods

This was a multicenter, randomized, placebo-controlled, double-blind, dose response study of intramuscular phenoxybenzamine hydrochloride (PBZ) versus vehicle and saline placebos. Phenoxybenzamine formulation known in the art (propylene glycol and alcohol) and diluted in saline at the time of injection were evaluated. The pH of these formulations was less than 2, and they were found to be very irritating. Patients with pain in the most commonly affected regions, the upper and lower back, were studied. Patients enrolled in this study were male or female with 4 months to 8 years of myofascial pain syndrome with identifiable active trigger points. Patients were required to have had EMG documented triggers points and meet the criteria of <10 uV in the adjacent muscle site and have a minimum of a 2:1 ratio of trigger point amplitude: adjacent amplitude. Attempts were made to obtain maximum amplitude in the trigger point.

The primary objectives of this study were to evaluate the safety, tolerance, and efficacy of 4 doses (0.1, 0.3, 1.0, or 2.4 mg) of phenoxybenzamine per intramuscular injection site. The vehicle placebo group received vehicle in a concentration equivalent to the active doses. A second placebo was saline. All injections were of 0.3 ml in volume. Up to 10 trigger points were injected at each treatment session. There were up to 2 treatment sessions up to 10 days apart. The primary efficacy evaluation was made at week 12 of treatment. The Visual Analogue Pain Scale (VAS) was used to assess pain in the affected region before and after treatment. Secondary efficacy assessments included the McGill Pain Questionnaire (MPQ), the Spinal Function Sort (SFS) and the SF-36 Quality of Life questionnaire.

Results

For the 1.0 mg group, only a 9.9% difference in percent change from baseline between placebo and active drug was achieved, which was not statistically significant. The 2.4 mg group was equivalent to placebo. Thus, dosages of 1.0 mg and 2.4 mg were formulated in a propylene glycol and alcohol composition, and these formulations appeared to be ineffective in this study.

The most frequent adverse event (77% of all events) is injection site pain. The pain is usually mild to moderate in severity and has a mean duration of less than 10 days. Both the severity and duration of this adverse event have decreases as more dilute solutions and lower dose of drug were used.

A total of 372 patients were enrolled and randomized at 22 centers. Duration of exposure is presented in Table 4.

TABLE 4
Patient Disposition by Treatment Group

| | Tmt Group | | | | |
|---|---|---|---|---|---|
| | | | | 8-week | 12-week |
| | Baseline | 1^{st} Tmt | 2^{nd} Tmt | FU | FU |
| Saline | 79 | 79 | 44 (56%) | 77 (97%) | 76 (96%) |
| Placebo | | (100%) | | | |
| Vehicle | 83 | 83 | 47 (57%) | 80 (96%) | 80 (96%) |
| Placebo | | | | | |
| 0.1 mg PBZ | 21 | 21 | 11 (52%) | 17 (81%) | 15 (71%) |
| 0.3 mg PBZ | 22 | 22 | 10 (45%) | 22 (100%) | 21 (95%) |
| 1.0 mg PBZ | 86 | 86 | 38 (44%) | 84 (98%) | 82 (95%) |
| 2.4 mg PBZ | 81 | 81 | 38 (47%) | 77 (95%) | 76 (94%) |

Adverse Events

Thirty-two of the 79 patients (41%) in the saline placebo group, 33 of 83 patients (40%) in the vehicle placebo group, 35 of the 86 patients (41%) in the 1 mg group, and 37 of the 81 patients (46%) in the 2.4 mg group experienced 1 or more treatment-emergent adverse experiences. For all treatment groups combined, the most common adverse experiences observed were dizziness: 8%; pain 5%; nausea: 3%, and sweating: 3%. Treatment-emergent adverse experiences that occurred in 2% or more of the total number of patients are displayed in Table 5. The most frequent adverse experiences were dizziness (8%) and pain (5%), followed by headache, sweating, and nausea (3% each).

TABLE 5
Number of Patients Reporting Treatment-Emergent Adverse
Experiences (occurring in 2% or more of the total number of patients)

| Body | | | | | | |
|---|---|---|---|---|---|---|
| System | | | | | | |
| Preferred | Pooled | 0.1 mg | 0.3 mg | 1 mg | 2.4 mg | |
| Term | Placebo | PBZ | PBZ | PBZ | PBZ | Total |
| TOTAL | 65 | 7 | 5 | 35 (41%) | 37 | 149 |
| NUMBER | (40%) | (33%) | (23%) | | (46%) | (40%) |
| OF | | | | | | |
| PATIENTS | | | | | | |
| Dizziness | 11 | 1 (5%) | 0 (0%) | 11 (13%) | 8 | 31 (8%) |
| | (7%) | | | | (10%) | |
| Pain | 4 (2%) | 1 (5%) | 0 (0%) | 7 (8%) | 5 (6%) | 17 (5%) |
| Headache | 5 (3%) | 0 (0%) | 1 (5%) | 5 (6%) | 0 (0%) | 11 (3%) |
| Sweating | 2 (1%) | 0 (0%) | 0 (0%) | 3 (3%) | 5 (6%) | 10 (3%) |
| Nausea | 4 (2%) | 0 (0%) | 1 (5%) | 1 (1%) | 4 (5%) | 10 (3%) |
| Injection | 0 (0%) | 0 (0%) | 0 (0%) | 2 (2%) | 7 (9%) | 9 (2%) |
| site edema | | | | | | |
| Syncope | 6 (4%) | 0 (0%) | 0 (0%) | 2 (2%) | 1 (1%) | 9 (2%) |
| Infection | 5 (3%) | 0 (0%) | 1 (5%) | 2 (2%) | 1 (1%) | 9 (2%) |
| Hypotension | 3 (2%) | 0 (0%) | 0 (0%) | 2 (2%) | 1 (1%) | 6 (2%) |
| Ecchymosis | 4 (2%) | 0 (0%) | 0 (0%) | 1 (1%) | 1 (1%) | 6 (2%) |
| Flu | 1 (1%) | 0 (0%) | 1 (5%) | 3 (3%) | 1 (1%) | 6 (2%) |
| syndrome | | | | | | |

Injection Site Soreness

Significantly more patients in the high dose groups experienced injection site soreness than patients in the placebo groups at the first and second treatments. Importantly; however, these results indicated even low dosages of phenoxybenzamine caused some adverse side effected at the injection site. This indicated that the vehicle used in this formulation can be improved to reduce side effects.

### Example III

### Preparation of Phenoxybenzamine Hydrochloride Compositions

Phenoxybenzamine HCI (PBA) and placebo solutions for IM injection were prepared and analyzed under cGMP conditions for use in clinical studies based on standard protocols developed by Pharmatek Laboratories, Inc.; Spectrum Chemical Mfg. Corp, Gardena Calif.; West Coast Analytical Services; and PrimaPharm of San Diego, Calif. Standard methods of preparation and qualitative analysis of the compositions described herein are well known in the art and is described in United States Pharmacopeia, USP 27/NF 22, National Publishing, 2004.

Standard batches of phenoxybenzamine hydrochloride injections can be prepared, for example, as a 10 mg/ml injection aliquot. Each ml of Phenoxybenzamine hydrochloride injection contains 10 mg of phenoxybenzamine hydrochloride, USP; and the total volume was brought up to 1.0 ml with polyethylene glycol 400 (e.g. PEG-400, NF). The exemplary 10 ml sample measured 400 daltons in molecular weight; 7.2 cs in viscosity at 210° F.; <0.05% residue on ignition; <5 ppm of heavy metals; 0.10% in ethylene and diethyleneglycol; 0.0 ug/g in free ethylene oxide; and 5 ug/g of free 1,4-dioxane. Quality control parameters, such as, for example, evaluation of potency based on chromatographic tracings, were conducted by the manufacturing site under current USP guidelines. In addition, the stability of an exemplary Phenoxybenzamine HCI solution was assessed and summarized in Table 6.

For determination of the pH value of the active solutions, a 5% solution of the Phenoxybenzamine HCL measured 3.25. Examples of packaging material include 2-mL vials, Pharmatek Lot No. 0001, Part No. PTC-0001; rubber stoppers, Pharmatek Lot No. 0002, Part No. PTC-0002; Truedge® seals, Pharmatek Lot No. 0003, Part No. PTC-0003; Supor® filters, Pharmatek Lot No. 0005, Part No. PTC-0004; and labels, to be generated by PrimaPharm Inc. These can be supplied in the form of a kit.

TABLE 6
GMP Stability Summary
Phenoxybenzamine HCl Solution (5 mg/0.5 mL) Lot 04RD06: 2-8° C. per Protocol 1035-006

| | | | | t = 3 | t = 6 | | |
|---|---|---|---|---|---|---|---|
| | | | t = 0 | Months | Months | t = 9 | t = 12 |
| | | | (25 Months | (28 Months | (31 Months | Months | Months |
| | | Release | @2-8° C.) | @2-8° C.) | @2-8° C.) | (34 Months | (37 Months |
| Parameter | Specification | Mar. 26, 2004 | May 2, 2006 | Aug. 4, 2006 | Nov. 6, 2006 | @2-8° C.) | @2-8° C.) |
| Appearance | Container and | Container | Container | Container | Container | | |
| | closue intact and | and closue | and closue | and closue | and closue | | |
| | secure. Clear, | intact and | intact and | intact and | intact and | | |
| | colorless | secure. | secure. | secure. | secure. | | |
| | solution at | Clear, | Clear, | Clear, | Clear, | | |
| | ambient | colorless | colorless | colorless | colorless | | |
| | temperature. | solution at | solution at | solution at | solution at | | |
| | White to off- | ambient | ambient | ambient | ambient | | |
| | white semi-solid | temperature. | temperature. | temperature. | temperature. | | |
| | at 2-8° C. | White to off- | White to off- | White to off- | White to off- | | |
| | | white semi- | white semi- | white semi- | white semi- | | |
| | | solid at 2-8° C. | solid at 2-8° C. | solid at 2-8° C. | solid at 2-8° C. | | |
| Identification | 1.00 ± 0.20 | 1.02 | 1.01 | 1.00 | 1.00 | | |
| Assay | 90.0-110.0% | 98.8 | 95.5 | 97.0 | 96.7 | | |
| % Largest | ≦2.0 | 0.5 | 0.3 | 0.5 | 0.5 | | |
| Impurity | | | | | | | |
| % of Total | ≦5.0 | 1.0 | 0.7 | 1.3 | 0.9 | | |
| Impurities | | | | | | | |
| pH of a 5% | 3.4 ± 1.0 | 3.3 | 3.2 | 3.2 | 3.2 | | |
| Solution | | | | | | | |

Drug Product has been stored at 2-8° C. prior to the initiation of this protocol.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A pharmaceutical composition comprising phenoxybenzamine or a salt thereof and a pharmaceutically acceptable polyethylene glycol that is a liquid at room temperature.

2. A pharmaceutical composition of claim 1 for intramuscular injection.

3. A pharmaceutical composition of claim 1 for intravenous injection.

4. A pharmaceutical composition of claim 1 suitable for injection in a human.

5. A pharmaceutical composition of claim 1 for local injection into an area of muscle associated with pain in a human.

6. A pharmaceutical composition of claim 1 for subcutaneous injection.

7. A pharmaceutical composition of claim 1 further comprising a buffer which further comprises an acid or base for the adjustment of the pH.

8. A pharmaceutical composition of claim 1 comprising less than about 2% water.

9. A pharmaceutical composition of claim 1 comprising less than about 1% water.

10. A pharmaceutical composition of claim 1 comprising about 0.5% water.

11. A pharmaceutical composition of claim 1 comprising at least 90% by volume of a polyethylene glycol.

12. A pharmaceutical composition of claim 1 comprising at least 95% by volume of a polyethylene glycol.

13. A pharmaceutical composition of claim 1 comprising at least 97% by volume of a polyethylene glycol.

14. A pharmaceutical composition of claim 1 comprising at least 99% by volume of a polyethylene glycol.

15. A pharmaceutical composition of claim 1 consisting essentially of phenoxybenzamine or a salt thereof and a polyethylene glycol.

16. A pharmaceutical composition of claim 1 comprising a mixture of two or more polyethylene glycols wherein each polyethylene glycol has a different molecular weight.

17. A pharmaceutical composition of claim 1 wherein said polyethylene glycol has a molecular weight of between about 300 daltons and about 500 daltons.

18. A pharmaceutical composition of claim 1 wherein said polyethylene glycol comprises polyethylene glycol 400.

19. A pharmaceutical composition of claim 1 comprising a mixture of polyethylene glycol 400 and another polyethylene glycol having a different molecular weight.

20. A pharmaceutical composition of 1 wherein said phenoxybenzamine is phenoxybenzamine hydrochloride.

21. A pharmaceutical composition of claim 1 as a dosage form.

22. A pharmaceutical composition of claim 1 as a single dosage form.

23. A pharmaceutical composition of claim 1 as a single dosage form suitable for intramuscular injection.

24. A pharmaceutical composition of claim 1 as a single dosage form comprising phenoxybenzamine in an amount of from about 0.3 mg to about 10 mg.

25. A pharmaceutical composition of claim 1 as a single dosage form comprising phenoxybenzamine in an amount of from about 3 mg to about 7 mg.

26. A pharmaceutical composition of claim 1 as a single dosage form comprising phenoxybenzamine in an amount of about 5 mg.

27. A pharmaceutical composition of claim 1 as a single dosage form comprising phenoxybenzamine in an amount of about 3 mg.

28. A pharmaceutical composition of claim 1 as a single dosage form in a unit dosage volume of between about 0.3 ml and about 3 ml.

29. A pharmaceutical composition of claim 1 as a single dosage form comprising phenoxybenzamine in an amount of between about 3 mg and about 7 mg in a unit dosage volume of between about 0.3 ml and about 0.7 ml.

30. A pharmaceutical composition of claim 1 as a single dosage form of about 5 mg in about 0.5 ml.

31. A pharmaceutical composition of claim 1 as a single dosage form of about 0.5 ml at a concentration of about 10 mg/ml.

32. A pharmaceutical composition of claim 1 as a single dosage form of about 0.3 ml at a concentration of about 10 mg/ml.

33. A pharmaceutical composition of any one of claims 22-28 for intramuscular injection.

34. A pharmaceutical composition of claim 1 which has a pH of from about 2 to about 4.

35. A pharmaceutical composition of claim 1 which has a pH of from about 3 to about 4.

36. A pharmaceutical composition of claim 1 which has a pH of from about 2.8 to about 3.8.

37. A pharmaceutical composition of claim 1 which has a pH of about 3 to about 3.5.

38. A pharmaceutical composition of claim 1 which further comprises an osmotic modulating agent.

39. A pharmaceutical composition of claim 1 which further comprises a preservative.

40. A pharmaceutical composition of claim 1 which further comprises a buffer.

41. A pharmaceutical composition of claim 1 which is formulated for optimal tissue distribution.

42. A pharmaceutical composition comprising phenoxybenzamine or a salt thereof and an oil that is a liquid at room temperature.

43. A pharmaceutical composition of claim 42 consisting essentially of a phenoxybenzamine or a salt thereof and an oil.

44. A pharmaceutical composition of claim 42 wherein said oil is selected from the group consisting of peanut oil, sesame oil, and cottonseed oil.

45. A method for reducing chronic pain, said method comprising locating at least one myofascial trigger point in a muscle; and contacting the myofascial trigger point with a pharmaceutical composition of claim 1, whereby said chronic pain is reduced.

46. A kit comprising one or more pharmaceutically acceptable containers comprising the pharmaceutical composition of any claims 1-45.

47. A kit comprising i) a pharmaceutical composition of any claims 1-45, ii) means for containing the pharmaceutical composition, and iii) means for injection the pharmaceutical composition into a human.
